(19) United States
(12) Patent Application Publication
(10) Pub. No.: US 2026/0254286 A1
(43) Pub. Date: Aug. 27, 2026

(54) External Charger for an Implantable Medical Device Employing Sense Coil Amplitude Ratio for Adjusting Power and Determining Alignment

(71) Applicant: Boston Scientific Neuromodulation Corporation, Valencia, CA (US)

(72) Inventors: Joey Chen, Stevenson Ranch, CA (US); Tom Stouffer, Chatsworth, CA (US); Jonathan Larcom, Santa Clarita, CA (US); Daniel Aghassian, La Canada, CA (US); Gaurav Gupta, Valencia, CA (US)

(21) Appl. No.: 19/387,001

(22) Filed: Nov. 12, 2025

Related U.S. Application Data
(60) Provisional application No. 63/764,364, filed on Feb. 27, 2025.

(51) Int. Cl. H02J 50/90; A61N 1/378; H02J 50/12

(52) U.S. Cl. CPC H02J 50/90; A61N 1/3787; H02J 50/12

(57) ABSTRACT

An external charger for an Implantable Medical Device (IMD) is disclosed having a primary charging coil and preferably two sense coils. The sense coils comprise an outer and inner sense coil, which are preferably concentric with the primary charging coil. An amplitude is induced on the sense coils, and a parameter such as ratio of these amplitudes is assessed by the charger to adjust the power of the magnetic field generated by the primary charging coil, or determine an alignment between the charging coil and the IMD, or both.

### CROSS REFERENCE TO RELATED APPLICATIONS

This is a non-provisional of U.S. Provisional Patent Application Serial No. 63/764,364, filed February 27, 2025, which is incorporated herein by reference in its entirety, and to which priority is hereby claimed.

### FIELD OF THE INVENTION

The present invention relates to wireless external chargers for use in charging implantable medical devices.

### INTRODUCTION

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Patent 6,516,227. However, the present invention may find applicability in any implantable medical device system.

As shown in FIGS. 1A-1C, an SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example. The conductive case 12 may also act as an electrode.

In an SCS application, the electrode leads 18 are typically implanted in the spinal column inside the patient’s vertebrae and proximate to the dura in a patient’s spinal cord, preferably spanning left and right of the patient’s spinal column. Proximal contacts on the leads 18 are tunneled through the patient’s tissue to a distant location such as the upper buttocks where the IMD case 12 is implanted, at which point they are coupled to the lead connectors 26. SCS therapy is traditionally used to relieve symptoms such as chronic back pain. IMD 10 as described should be understood as including non-implantable External Trial Stimulators (ETSs), which mimic operation of the IMD 10 during trials periods when electrode array 20 has been implanted in the patient but the IMD has not. See, e.g., USP 9,259,574 (disclosing an ETS).

FIGS. 1B and 1C show plan and cross-sectional views of the IMD 10, with the case 12 removed in FIG. 1B for easier viewing of internal components. As shown, the IMD 10 typically includes a printed circuit board (PCB) 30, along with various electronic components 32 mounted to the PCB 30, some of which are shown in FIG. 3. Two coils (more generally, antennas) are shown in the IMD 10: a telemetry coil 34 used to transmit/receive data to/from an external controller (not shown); and a charging coil 36 for charging or recharging the IMD’s battery 14 using an external charger. These coils 34 operate on a principle of magnetic induction to both receive power from an external charger and to communicate bidirectionally with an external controller (not shown; see e.g. USP 11,559,693). Although not shown, the IMD 10 can also include a short-range RF antenna in lieu of telemetry coil 34 which can communicate with the external controller or the external charger using far-field electromagnetic waves, using the well-known Bluetooth standard for example.

FIG. 2A shows a plan view of a traditional external charger 50 for the IMD 10, while FIG. 2B shows the external charger 50 and IMD 10 in cross section with the external charger 50 wirelessly conveying power via a magnetic field 45 to the IMD 10, which power can be used to operate the IMD and/or recharge the IMD’s battery 14. The external charger 50 contains one or more PCB 54 on which electronic components 56 are placed, see USP 9,002,445, some of which are shown in FIG. 3. A user interface 58 including an on/off switch 60 allows a patient or clinician to operate the external charger 50 to start and stop generation of the magnetic field 45. User interface 58 may also include a Light Emitting Diode (LEDs) or other lamps and possibly also a speaker to indicate status. A battery 64 provides power for the external charger 50, which battery 64 may itself be rechargeable. The external charger 50 can also receive AC power from a wall plug for power, or to charge the battery 64. The charger 50 may also include a temperature sensor 68 to monitor the temperature during charging. If the temperature gets too hot (above a threshold), thus risking patent discomfort or injury, the charger 50 can be controlled to temporarily suspend producing the magnetic field 45 until the temperature drops to a safe level. In this regard, operation of the charger may duty cycle on and off, unbeknownst to the user. A hand-holdable housing 66 sized to fit a user’s hand contains all of the charger’s components.

Power transmission from the external charger 50 to the IMD 10 occurs wirelessly and transcutaneously through a patient’s tissue 25 via inductive coupling, and FIG. 3 shows details of the circuitry used to implement such functionality. Primary charging coil 52 in the external charger 50 is energized via charging circuit 70 with an AC current, Icharge, to create the AC magnetic field 45. This magnetic field 45 induces a current in the secondary charging coil 36 within the IMD 10, providing a voltage across coil 36 that is rectified (38) to DC levels and used to recharge the battery 14, either by application of a battery charging current Ibat or by application of a voltage to the battery. This voltage or current can be regulated by charging and protection circuitry 40 as shown. The frequency of the magnetic field 45 (and AC current Icharge) can be perhaps 80 kHz or so. When charging the battery 14 in this manner, it is typical that the housing 66 of the external charger 50 touches the patient’s tissue 25, perhaps with a charger holding device or the patient’s clothing intervening, although this is not strictly necessary. The charger 50 and IMD 10 can respectively include control circuitries 72 and 42 to control operation of charging and other functions in these devices.

The IMD 10 can send telemetry to the charger 50, and in the example shown in FIG. 3 this occurs using Load Shift Keying (LSK). LSK can generally be used to send any sort of digital data (LSK data) to the charger 50, including data that can be used to control charger 50 functionality. For example, during a charging session, the IMD 10 could LSK telemeter the temperature of the IMD 10 (as sensed by a temperature sensor in the IMD 10), the current voltage of the IMD 10’s battery 14 (Vbat, which will increase as the battery charges), or the charging current Ibat that battery is receiving, all of which may be relevant to adjusting the power of the magnetic field 45 or turning it off.

In the example shown, LSK data telemetered from the IMD 10 is limited to transmission of an end-of-charge (EoC) signal, which informs the charger 50 to stop producing the magnetic field 45. The EoC signal can be sent in a number of different circumstances. Primarily, the EoC signal is transmitted when the IMD 10 determines that its battery 14 is full, and therefore that charging is no longer needed. This determination can be made by comparing the battery voltage, Vbat, to a threshold in the IMD’s control circuitry 42. However, the EoC signal can also be sent for example if the IMD’s temperature (T, as sensed by an IMD temperature sensor) is too high (again, above a threshold) during a charging session.

An LSK modulator 74 in the IMD 10 is enabled when the EoC signal (or other LSK data) is to be transmitted. As shown, the LSK modulator 74 may comprise a firmware module in the IMD’s control circuitry 42. The LSK modulator 74 outputs a series of digital data bits each with a bit duration td. While this series of bits could be more random (e.g., if more detailed LSK data like Ibat, Vbat, or T is being sent), in the depicted example the EoC signal comprises a sequence of a specific number (e.g., 128) of alternating bits (010101…). This signal is sent to the gates of switches 76, thus alternatively opening (0) and closing (1) them, which causes the ends of the secondary coil 36 in the IMD 10 to be either floating (as they are normally when receiving the magnetic field 45) or grounded.

Repeatedly grounding and floating the ends of the coil 36 via the EoC signal affects the mutual impedance between the charger 50 and the IMD 10, and in particular the mutual impedance between the primary charging coil 52 and the secondary charging coil 36. More specifically, this change in mutual impedance causes a reflected impedance which is detectable at the charger 50. Assuming that AC current Icharge through the primary charging coil 52 is of a constant magnitude, the reflected impedance will cause the magnitude of the AC voltage across this coil, Vcoil, to change, with this voltage being lower when the secondary coil 36 is floating (when EoC=0), and higher when that coil is grounded (when EoC=1).

Thus, this coil voltage Vcoil across the primary charging coil 52 can be assessed to recover the transmitted LSK data. This occurs by rectifying (78) the magnitude of Vcoil to a DC voltage, Vcoil(dc), and providing this voltage to a LSK demodulator 80. The LSK demodulator 80 monitors for changes in the magnitude of Vcoil(dc), and assesses whether such changes occur in accordance with the bit duration (td) and number of bits (e.g., 128) in the EoC signal. If the LSK demodulator 80 upon assessment of Vcoil(dc) detects receipt of the EoC signal, the LSK demodulator 80 informs the control circuitry 72 in the charger to cease producing Icharge through the primary charging coil 52, thus ceasing production of the magnetic field 45. Digital portions of the LSK demodulator 80 may be included as part of the charger’s control circuitry 72.

The IMD 10 may take other steps after transmitting an EoC signal. For example, after transmitting the EoC signal, the IMD 10 may decouple (e.g., ground via switches 76) its secondary coil 36 for some time to prohibit further receipt of the magnetic field 45.

### SUMMARY

An external charger for wirelessly providing energy to an implantable medical device (IMD) is disclosed, which may comprise: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; and use the ratio to adjust a power of the magnetic field.

The external charger can include any of the following modifications or additions in any combination. In one example, control circuitry is further configured to determine an alignment between the charging coil and the IMD using the ratio. In one example, the control circuitry is configured to determine the alignment by determining if the charging coil and the IMD are aligned or misaligned by comparing the ratio to a threshold. In one example, the control circuitry is configured to determine that the charging coil and the IMD are misaligned when the ratio is less than the threshold. In one example, the external charger further comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger further comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the ratio periodically during the charging session, and thus periodically use the ratio to adjust the power of the magnetic field. In one example, the external charger comprises a first housing comprising the charging coil and the first and second sense coils, a second housing comprising the control circuitry, and a cable for passing signals between the first and second housings. In one example, the external charger further comprises an amplifier, wherein the amplifier is configured to energize the charging coil to produce the magnetic field in accordance with a drive signal. In one example, the drive signal comprises a duty cycle, and wherein the control circuitry is configured to use the ratio to adjust the power of the magnetic field by using the ratio to adjust the duty cycle of the drive signal. In one example, the control circuitry comprises a pulse width modulator to adjust the duty cycle of the drive signal. In one example, the control circuitry further comprises a function relating ratios to duty cycles, and wherein the control circuitry is configured to adjust the duty cycle using the ratio and the function. In one example, the first sense coil is larger than the second sense coil, and wherein the function proportionately relates the ratios to the duty cycles. In one example, the first and second induced signals are AC signals, and wherein the first and second amplitudes are respectively indicative of maximum amplitudes of the first and second induced signals.

A method for wirelessly providing energy to an implantable medical device (IMD) is disclosed, which may comprise: energizing a charging coil in an external charger to produce a magnetic field to wirelessly provide energy to the IMD during a charging session; inducing by the magnetic field a first induced signal on a first sense coil in the external charger; inducing by the magnetic field a second induced signal on a second sense coil in the external charger; determining a first amplitude of the first induced signal and a second amplitude of the second induced signal; dividing the first amplitude by the second amplitude to determine a ratio; and using the ratio to adjust a power of the magnetic field.

The method can include any of the following modifications or additions in any combination. In one example, the method further comprises determining an alignment between the charging coil and the IMD using the ratio. In one example, the charging coil and the IMD are determined to be aligned or misaligned by comparing the ratio to a threshold. In one example, the charging coil and the IMD are determined to be misaligned when the ratio is less than the threshold. In one example, the external charger comprises a user interface comprising one or more user interface elements, wherein if the charging coil and the IMD are determined to be misaligned, one or more of the user interface elements is enabled. In one example, the one or more user interface elements comprise one or more of a visual indicator or a speaker. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the ratio is periodically determined during the charging session, and thus the ratio is used to adjust the power of the magnetic field periodically during the charging sessions. In one example, the external charger comprises a first housing comprising the charging coil and the first and second sense coils, a second housing, and a cable for passing signals between the first and second housings. In one example, an amplifier is used to energize the charging coil to produce the magnetic field in accordance with a drive signal. In one example, the drive signal comprises a duty cycle, and wherein the ratio is used to adjust the power of the magnetic field by using the ratio to adjust the duty cycle of the drive signal. In one example, the duty cycle of the drive signal is adjusted using a pulse width modulator in the external charger. In one example, the external charger comprises a function relating ratios to duty cycles, and wherein the duty cycle is adjusted using the ratio and the function. In one example, the first sense coil is larger than the second sense coil, and wherein the function proportionately relates the ratios to the duty cycles. In one example, the first and second induced signals are AC signals, and wherein the first and second amplitudes are respectively indicative of maximum amplitudes of the first and second induced signals.

A system is disclosed, which may comprise: an implantable medical device (IMD); and an external charger for wirelessly providing energy to the IMD; the external charger comprising: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; and use the ratio to adjust a power of the magnetic field.

The system can include any of the following modifications or additions in any combination. In one example, the control circuitry is further configured to determine an alignment between the charging coil and the IMD using the ratio. In one example, the control circuitry is configured to determine the alignment by determining if the charging coil and the IMD are aligned or misaligned by comparing the ratio to a threshold. In one example, the control circuitry is configured to determine that the charging coil and the IMD are misaligned when the ratio is less than the threshold. In one example, the external charger comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the control circuitry is configured to determine the ratio periodically during the charging session, and thus periodically use the ratio to adjust the power of the magnetic field. In one example, the external charger comprises a first housing comprising the charging coil and the first and second sense coils, a second housing comprising the control circuitry, and a cable for passing signals between the first and second housings. In one example, the external charger comprises an amplifier, wherein the amplifier is configured to energize the charging coil to produce the magnetic field in accordance with a drive signal. In one example, the drive signal comprises a duty cycle, and wherein the control circuitry is configured to use the ratio to adjust the power of the magnetic field by using the ratio to adjust the duty cycle of the drive signal. In one example, the control circuitry comprises a pulse width modulator to adjust the duty cycle of the drive signal. In one example, the control circuitry further comprises a function relating ratios to duty cycles, and wherein the control circuitry is configured to adjust the duty cycle using the ratio and the function. In one example, the first sense coil is larger than the second sense coil, and wherein the function proportionately relates the ratios to the duty cycles. In one example, the first and second induced signals are AC signals, and wherein the first and second amplitudes are respectively indicative of maximum amplitudes of the first and second induced signals. In one example, the IMD comprises an implantable neurostimulator device or an inflatable penile implant.

A computer readable medium is disclosed, which may comprise instructions executable in an external charger for wirelessly providing energy to an implantable medical device (IMD). The instructions when executed are configured to energize a charging coil in the external charger to produce a magnetic field to wirelessly provide energy to the IMD during a charging session; determine a first amplitude of a first induced signal induced on a first sense coil in the external charger and a second amplitude of a second induced signal induced on a second sense coil in the external charger; divide the first amplitude by the second amplitude to determine a ratio; and use the ratio to adjust a power of the magnetic field.

An external charger for wirelessly providing energy to an implantable medical device (IMD) is disclosed, which may comprise: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; and use the ratio to determine an alignment between the charging coil and the IMD and to adjust a power of the magnetic field.

The external charger can include any of the following modifications or additions in any combination. In one example, the control circuitry is configured to determine the alignment by determining if the charging coil and the IMD are aligned or misaligned by comparing the ratio to a threshold. In one example, the control circuitry is configured to determine that the charging coil and the IMD are misaligned when the ratio is less than the threshold. In one example, the external charger further comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger further comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the ratio periodically during the charging session, and thus periodically use the ratio to determine the alignment between the charging coil and the IMD. In one example, the external charger comprises a first housing comprising the charging coil and the first and second sense coils, a second housing comprising the control circuitry, and a cable for passing signals between the first and second housings. In one example, the external charger further comprises an amplifier, wherein the amplifier is configured to energize the charging coil to produce the magnetic field in accordance with a drive signal. In one example, the control circuitry is configured to use the ratio to adjust the power of the magnetic field by using the ratio to adjust the duty cycle of the drive signal. In one example, the control circuitry further comprises a function relating ratios to duty cycles, and wherein the control circuitry is configured to adjust the duty cycle using the ratio and the function. In one example, the first sense coil is larger than the second sense coil, and wherein the function proportionately relates the ratios to the duty cycles. In one example, the control circuitry is configured to use the ratio to adjust the power of the magnetic field only if the control circuitry first determines that the charging coil and the IMD are not misaligned. In one example, the first and second induced signals are AC signals, and wherein the first and second amplitudes are respectively indicative of maximum amplitudes of the first and second induced signals.

A method for wirelessly providing energy to an implantable medical device (IMD) is disclosed, which may comprise: energizing a charging coil in an external charger to produce a magnetic field to wirelessly provide energy to the IMD during a charging session; inducing by the magnetic field a first induced signal on a first sense coil in the external charger; inducing by the magnetic field a second induced signal on a second sense coil in the external charger; determining a first amplitude of the first induced signal and a second amplitude of the second induced signal; dividing the first amplitude by the second amplitude to determine a ratio; and using the ratio to determine an alignment between the charging coil and the IMD and to adjust a power of the magnetic field.

The method can include any of the following modifications or additions in any combination. In one example, the alignment is determined by determining if the charging coil and the IMD are aligned or misaligned by comparing the ratio to a threshold. In one example, the charging coil and the IMD are determined to be misaligned when the ratio is less than the threshold. In one example, the external charger comprises a user interface comprising one or more user interface elements, wherein if the control circuitry determines that the charging coil and the IMD are misaligned, one or more of the user interface elements is enabled. In one example, the one or more user interface elements comprise one or more of a visual indicator or a speaker. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the ratio is determined periodically during the charging session, and thus the ratio is used to determine the alignment between the charging coil and the IMD periodically and to adjust the power of the magnetic field periodically during the charging session. In one example, the external charger comprises a first housing comprising the charging coil and the first and second sense coils, a second housing, and a cable for passing signals between the first and second housings. In one example, the external charger comprises an amplifier, wherein the amplifier energizes the charging coil to produce the magnetic field in accordance with a drive signal. In one example, the ratio is used to adjust the power of the magnetic field by using the ratio to adjust the duty cycle of the drive signal. In one example, the external charger comprises a function relating ratios to duty cycles, and wherein the duty cycle is adjusted using the ratio and the function. In one example, the first sense coil is larger than the second sense coil, and wherein the function proportionately relates the ratios to the duty cycles. In one example, the ratio is used to adjust the power of the magnetic field only if the charging coil and the IMD are determined to not be misaligned. In one example, the first and second induced signals are AC signals, and wherein the first and second amplitudes are respectively indicative of maximum amplitudes of the first and second induced signals.

A system is disclosed, which may comprise: an implantable medical device (IMD); and an external charger for wirelessly providing energy to the IMD, the external charger comprising: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; and use the ratio to determine an alignment between the charging coil and the IMD and to adjust a power of the magnetic field.

The system can include any of the following modifications or additions in any combination. In one example, the control circuitry is configured to determine the alignment by determining if the charging coil and the IMD are aligned or misaligned by comparing the ratio to a threshold. In one example, the control circuitry is configured to determine that the charging coil and the IMD are misaligned when the ratio is less than the threshold. In one example, the external charger comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the ratio periodically during the charging session, and thus periodically use the ratio to determine the alignment between the charging coil and the IMD. In one example, the external charger comprises a first housing comprising the charging coil and the first and second sense coils, a second housing comprising the control circuitry, and a cable for passing signals between the first and second housings. In one example, the external charger comprises an amplifier, wherein the amplifier is configured to energize the charging coil to produce the magnetic field in accordance with a drive signal. In one example, the control circuitry is configured to use the ratio to adjust the power of the magnetic field by using the ratio to adjust the duty cycle of the drive signal. In one example, the control circuitry further comprises a function relating ratios to duty cycles, and wherein the control circuitry is configured to adjust the duty cycle using the ratio and the function. In one example, the first sense coil is larger than the second sense coil, and wherein the function proportionately relates the ratios to the duty cycles. In one example, the control circuitry is configured to use the ratio to adjust the power of the magnetic field only if the control circuitry first determines that the charging coil and the IMD are not misaligned. In one example, the first and second induced signals are AC signals, and wherein the first and second amplitudes are respectively indicative of maximum amplitudes of the first and second induced signals. In one example, the IMD comprises an implantable neurostimulator device or an inflatable penile implant.

A computer readable medium is disclosed, which may comprise instructions executable in an external charger for wirelessly providing energy to an implantable medical device (IMD). The instructions when executed are configured to energize a charging coil in the external charger to produce a magnetic field to wirelessly provide energy to the IMD during a charging session; determine a first amplitude of a first induced signal induced on a first sense coil in the external charger and a second amplitude of a second induced signal induced on a second sense coil in the external charger; divide the first amplitude by the second amplitude to determine a ratio; and use the ratio to determine an alignment between the charging coil and the IMD and to adjust a power of the magnetic field.

An external charger for wirelessly providing energy to an implantable medical device (IMD) is disclosed, which may comprise: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; use the ratio to determine if the charging coil and the IMD are aligned or misaligned; and wherein if the charging coil and the IMD are aligned, the control circuitry is further configured to adjust a power of the magnetic field using the ratio.

The external charger can include any of the following modifications or additions in any combination. In one example, the control circuitry is configured to determine if the charging coil and the IMD are aligned or misaligned by comparing the ratio to a threshold. In one example, the control circuitry is configured to determine that the charging coil and the IMD are misaligned when the ratio is less than the threshold. In one example, the external charger further comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger further comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the ratio periodically during the charging session, and thus periodically use the ratio to determine if the charging coil and the IMD are aligned or misaligned and to adjust the power of the magnetic field. In one example, the external charger comprises a first housing comprising the charging coil and the first and second sense coils, a second housing comprising the control circuitry, and a cable for passing signals between the first and second housings. In one example, the external charger further comprises an amplifier, wherein the amplifier is configured to energize the charging coil to produce the magnetic field in accordance with a drive signal, wherein the drive signal comprises a duty cycle, and wherein the control circuitry is configured to use the ratio to adjust the power of the magnetic field by using the ratio to adjust the duty cycle of the drive signal. In one example, the first and second induced signals are AC signals, and wherein the first and second amplitudes are respectively indicative of maximum amplitudes of the first and second induced signals. In one example, if the charging coil and the IMD are aligned, the control circuitry is further configured to adjust a frequency of the magnetic field. In one example, the control circuitry is configured to adjust the power of the magnetic field at one or more times that are different from one or more times when the frequency is adjusted. In one example, the control circuitry is configured to interleave the one or more times when the power is adjusted with the one or more times when the frequency is adjusted. In one example, the external charger further comprises an amplifier, wherein the amplifier is configured to energize the charging coil to produce the magnetic field in accordance with a drive signal, wherein the control circuitry comprises a pulse width modulator to adjust a duty cycle of the drive signal. In one example, the control circuitry is configured to adjust the frequency of the magnetic field by programming the pulse width modulator to adjust the duty cycle of the drive signal, and wherein the control circuitry is configured to adjust the power of the magnetic field by programming the pulse width modulator to adjust the duty cycle of the drive signal.

A method for wirelessly providing energy to an implantable medical device (IMD) is disclosed, which may comprise: energizing a charging coil in an external charger to produce a magnetic field to wirelessly provide energy to the IMD during a charging session; inducing by the magnetic field a first induced signal on a first sense coil in the external charger; inducing by the magnetic field a second induced signal on a second sense coil in the external charger; determining a first amplitude of the first induced signal and a second amplitude of the second induced signal; dividing the first amplitude by the second amplitude to determine a ratio; and using the ratio to determine if the charging coil and the IMD are aligned or misaligned, and only if the charging coil and the IMD are aligned, using the ratio to adjust a power of the magnetic field.

The method can include any of the following modifications or additions in any combination. In one example, the charging coil and the IMD are determined to be aligned or misaligned by comparing the ratio to a threshold. In one example, the charging coil and the IMD are determined to be misaligned when the ratio is less than the threshold. In one example, the external charger comprises a user interface comprising one or more user interface elements, wherein if the charging coil and the IMD are determined to be misaligned, one or more of the user interface elements is enabled. In one example, the one or more user interface elements comprise one or more of a visual indicator or a speaker. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the ratio is determined periodically during the charging session, and thus the ratio is used to periodically determine if the charging coil and the IMD are aligned or misaligned or to adjust the power of the magnetic field during the charging session. In one example, the external charger comprises a first housing comprising the charging coil and the first and second sense coils, a second housing, and a cable for passing signals between the first and second housings. In one example, the external charger comprises an amplifier, wherein the amplifier energizes the charging coil to produce the magnetic field in accordance with a drive signal, wherein the drive signal comprises a duty cycle, and wherein the ratio is used to adjust the power of the magnetic field by using the ratio to adjust the duty cycle of the drive signal. In one example, the first and second induced signals are AC signals, and wherein the first and second amplitudes are respectively indicative of maximum amplitudes of the first and second induced signals. In one example, if the charging coil and the IMD are determined to be aligned, further comprising adjusting a frequency of the magnetic field. In one example, the power of the magnetic field is adjusted at one or more times that are different from one or more times when the frequency is adjusted. In one example, the one or more times when the power is adjusted is interleaved with the one or more times when the frequency is adjusted. In one example, the external charger comprises an amplifier, wherein the amplifier energizes the charging coil to produce the magnetic field in accordance with a drive signal, wherein the external charger comprises a pulse width modulator to adjust a duty cycle of the drive signal. In one example, the frequency of the magnetic field is adjusted by programming the pulse width modulator to adjust the duty cycle of the drive signal, and wherein the power of the magnetic field is adjusted by programming the pulse width modulator to adjust the duty cycle of the drive signal.

A system is disclosed, which may comprise: an implantable medical device (IMD); and an external charger for wirelessly providing energy to the IMD, the external charger comprising: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a first amplitude of the first induced signal and a second amplitude of the second induced signal; divide the first amplitude by the second amplitude to determine a ratio; use the ratio to determine if the charging coil and the IMD are aligned or misaligned; and wherein if the charging coil and the IMD are aligned, the control circuitry is further configured to adjust a power of the magnetic field using the ratio.

The system can include any of the following modifications or additions in any combination. In one example, the control circuitry is configured to determine if the charging coil and the IMD are aligned or misaligned by comparing the ratio to a threshold. In one example, the control circuitry is configured to determine that the charging coil and the IMD are misaligned when the ratio is less than the threshold. In one example, the external charger comprises a user interface comprising one or more user interface elements, wherein if the control circuitry determines that the charging coil and the IMD are misaligned, the control circuitry is further configured to enable one or more of the user interface elements. In one example, the one or more user interface elements comprise one or more of a visual indicator or a speaker. In one example, the external charger comprises a circuit board comprising the charging coil, the first sense coil, and the second sense coil, wherein the charging coil, the first sense coil, and the second sense coil are concentric. In one example, the charging coil comprises a wire winding affixed to a side of the circuit board. In one example, the first and second sense coils comprise one or more traces in the circuit board. In one example, the first sense coil has a radius larger than a radius of the second sense coil. In one example, the charging coil has a radius larger than the radii of the first and second sense coils. In one example, the control circuitry is configured to determine the ratio periodically during the charging session, and thus periodically use the ratio to determine if the charging coil and the IMD are aligned or misaligned and to adjust the power of the magnetic field. In one example, the external charger comprises a first housing comprising the charging coil and the first and second sense coils, a second housing comprising the control circuitry, and a cable for passing signals between the first and second housings. In one example, the external charger comprises an amplifier, wherein the amplifier is configured to energize the charging coil to produce the magnetic field in accordance with a drive signal, wherein the drive signal comprises a duty cycle, and wherein the control circuitry is configured to use the ratio to adjust the power of the magnetic field by using the ratio to adjust the duty cycle of the drive signal. In one example, the first and second induced signals are AC signals, and wherein the first and second amplitudes are respectively indicative of maximum amplitudes of the first and second induced signals. In one example, if the charging coil and the IMD are aligned, the control circuitry is further configured to adjust a frequency of the magnetic field. In one example, the control circuitry is configured to adjust the power of the magnetic field at one or more times that are different from one or more times when the frequency is adjusted. In one example, the control circuitry is configured to interleave the one or more times when the power is adjusted with the one or more times when the frequency is adjusted. In one example, the external charger comprises an amplifier, wherein the amplifier is configured to energize the charging coil to produce the magnetic field in accordance with a drive signal, wherein the control circuitry comprises a pulse width modulator to adjust a duty cycle of the drive signal. In one example, the control circuitry is configured to adjust the frequency of the magnetic field by programming the pulse width modulator to adjust the duty cycle of the drive signal, and wherein the control circuitry is configured to adjust the power of the magnetic field by programming the pulse width modulator to adjust the duty cycle of the drive signal. In one example, the IMD comprises an implantable neurostimulator device or an inflatable penile implant.

A computer readable medium is disclosed, which may comprise instructions executable in an external charger for wirelessly providing energy to an implantable medical device (IMD). The instructions when executed are configured to energize a charging coil in the external charger to produce a magnetic field to wirelessly provide energy to the IMD during a charging session; determine a first amplitude of a first induced signal induced on a first sense coil in the external charger and a second amplitude of a second induced signal induced on a second sense coil in the external charger; divide the first amplitude by the second amplitude to determine a ratio; and use the ratio to determine if the charging coil and the IMD are aligned or misaligned, and only if the charging coil and the IMD are aligned, use the ratio to adjust a power of the magnetic field.

### BRIEF DESCRIPTION OF THE DRAWINGS

FIGS. 1A-1C show different views of an implantable pulse generator, a type of implantable medical device (IMD), in accordance with the prior art.

FIG. 2A shows an external charger in accordance with the prior art, and FIG. 2B shows this charger being used to charge a battery in an IMD.

FIG. 3 shows basic circuitry in both the external charger and the IMD, in accordance with the prior art.

FIGS. 4A and 4B show an improved external charger having an electronics module and charging coil assembly connected by a cable.

FIGS. 5A and 5B show top and bottom sides of a circuit board in the charging coil assembly, which circuit board includes a primary charging coil, and outer and inner sense coils.

FIG. 6 shows signaling between the charging coil assembly and the electronics module in the charger.

FIG. 7 shows the amplifier circuitry for driving the coil in the charging coil assembly, and further shows various firmware modules within the control circuitry of the charger, including an alignment and power module for executing a power adjustment and alignment algorithm.

FIGS. 8A-8D show various positionings between the charging coil assembly and the IMD being charged, some of which indicate poor coupling and poor alignment.

FIG. 9 shows the drive signal for driving the primary charging coil, and signal induced on the outer and inner sense coils, including the amplitude of the induced signals.

FIG. 10 shows simulation data indicating the amplitudes of the induced signals, and their ratio R, as a function of charging coil assembly-to-IMD distance.

FIG. 11 shows operation of the power adjustment and alignment algorithm, which uses R to adjust the power and determine misalignment between the charging coil and the IMD.

FIG. 12 shows an example of another type of implantable medical device able to be charged by the disclosed charger.

### DETAILED DESCRIPTION

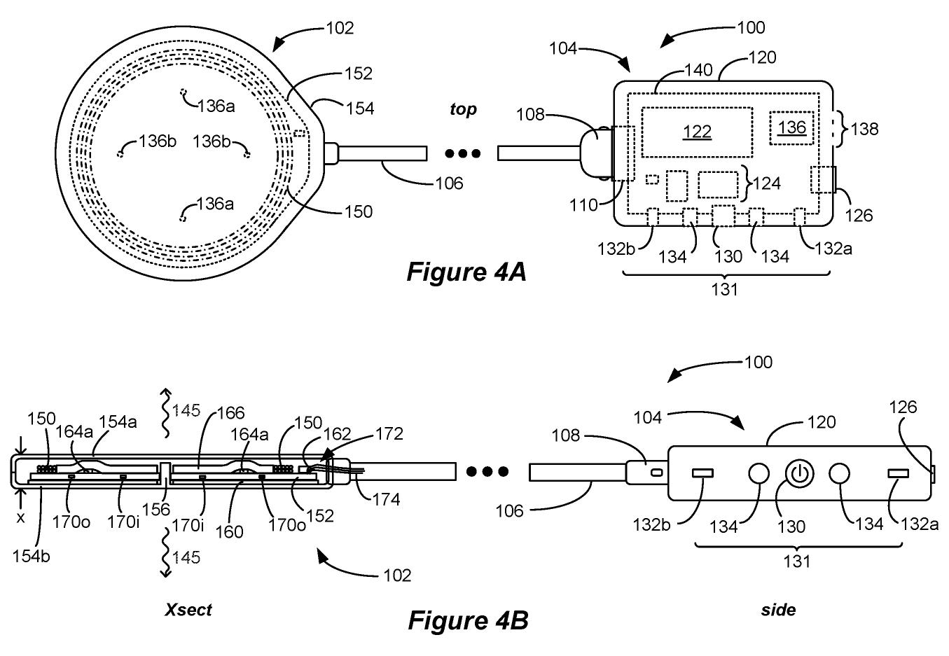

FIGS. 4A and 4B show a different design for an external charger 100 for an IMD 10, which is shown in a plan view (FIG. 4A) and in a side and cross-sectional view (FIG. 4B). The charger 100 is generally similar in structure to the charging systems disclosed in U.S. Patent Application Publication 2017/0361113, and U.S. Provisional Patent Application Serial No. 63/764,350, filed February 27, 2025. These patent applications are incorporated herein by reference in their entireties.

Charger 100 includes two main pieces: an electronics module 104 and a charging coil assembly 102 which includes a primary charging coil 150 used to produce a magnetic field 145 to charge the IMD 10’s battery 14, as explained earlier. The electronics module 104 and the charging coil assembly 102 are connected by a cable 106. The cable 106 may be separable from both the electronics module 104 and the charging coil assembly 102 via a port/connector arrangement, but as illustrated cable 106 is permanently affixed to the charging coil assembly 102. The other end of the cable 106 includes a connector 108 that can attach to and detach from a port 110 of the electronics module 104, although this end may be permanently affixed as well. The electronics module 104 preferably has a user interface 131, which is explained further below.

Separating the charging coil assembly 102 and the electronics module 104 via a cable 106 of significant length provides convenience for the patient, especially when compared to the single-housing design of charger 50 described earlier. For example, in a Spinal Cord Stimulation application where the IMD 10 is implanted behind the patient, the charging coil assembly 102 can be placed over the IMD 10 behind the patient, with the electronics module 104 looped around the patient’s waist and positioned in front of the patient where its user interface 131 can be more conveniently seen and accessed. Although not shown, a charging belt can be used to hold the charging coil assembly 102 and the electronics module 104 in convenient positions relative to the patient. This belt design can differ based upon the location at which the IMD 10 is implanted in the patient (e.g., Spinal Cord Stimulation v. Deep Brain Stimulation).

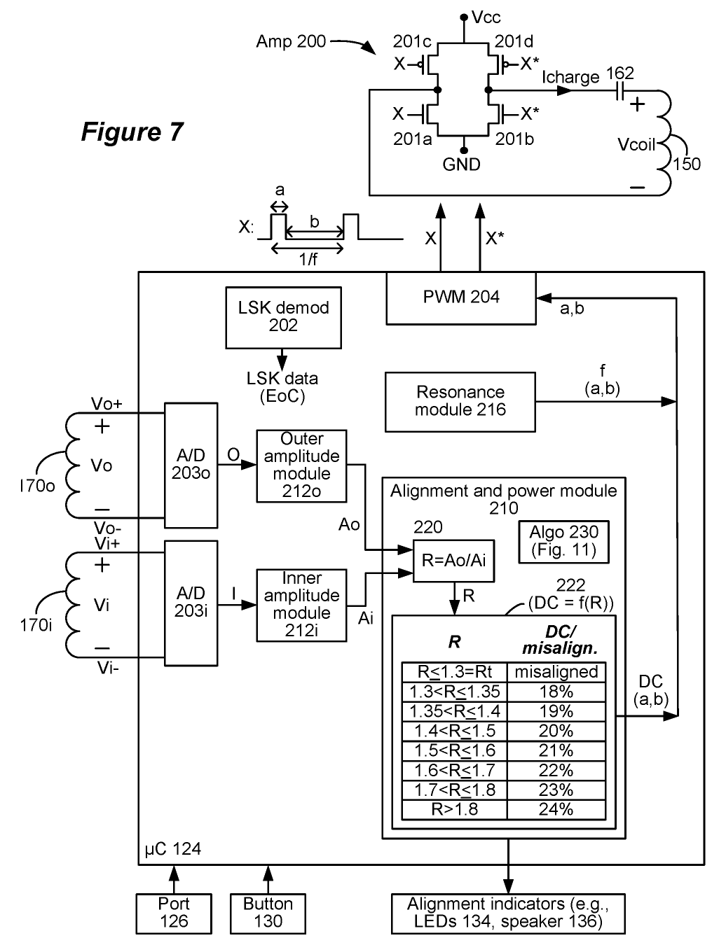

Electronics module 104 preferably includes within its housing 120 a battery 122 and control circuitry 124 needed for charging system operation. Control circuitry 124 is described in detail later with reference to FIG. 7, and can comprise a microcontroller programmed with firmware, such as any of the STM32L4 ARM series of microcontrollers provided by STMicroeletronics, Inc., as described at http:// www.st.com/ content/ st_com/ en/ products/ microcontrollers/ stm32-32-bit-arm-cortex-mcus/ stm32l4-series.html? querycriteria = productId=SS1580. Control circuitry 124 may also comprise an FPGA, DSP, or other similar digital logic devices. Control circuitry 124 can further comprise a memory programmed with firmware and accessible to a microcontroller or other digital logic device should that logic device not contain suitable on-chip memory. Control circuitry 124 may comprise a number of discrete components, and can comprise various analog circuitry and sensors in addition to digital processing logic. Electronics in the electronics module can be integrated via a circuit board 140. The electronics module 104 may further include a port 126 (e.g., a USB port) to allow its battery 122 to be recharged in conventional fashion, and/or to allow data to be read from or programmed into the electronics module, such as new operating software.

Housing 120 may also carry user interface elements 131, as shown in the side view of FIG. 4B. These elements can include a button 130, and one or more visual indicators such as LEDs 132a, 132b, and 134. The button 130 can be used to start and stop generation of the magnetic field 145 from the primary charging coil 126. As noted in the above-incorporated ‘350 Provisional Application, button 130 can also be used to adjust the power mode at which the charger operates, for example, in a low- or high-power mode. The button 130 may also be backlit by an LED, which could be lit for different purposes. For example, it could be lit to indicate that the charger 100 has been turned on and is producing a magnetic field 145. Alternatively, the LED associated with button 130 could be lit (e.g., green) to indicate good alignment between the charging coil assembly 102 and the IMD 10.

In one example, LED 132a is used to indicate the power status of the electronics module 104, i.e., the status (capacity) of the battery 122 and whether it needs charging. Charger battery LED 132a can for example be lit green, yellow, and red to indicate the (diminishing) capacity of the charger’s battery 122 based on measurements taken by the control circuitry 124 (such as the voltage of the battery 122), and can blink red when the charger battery 122 is critically low and recharging is required (via port 126).

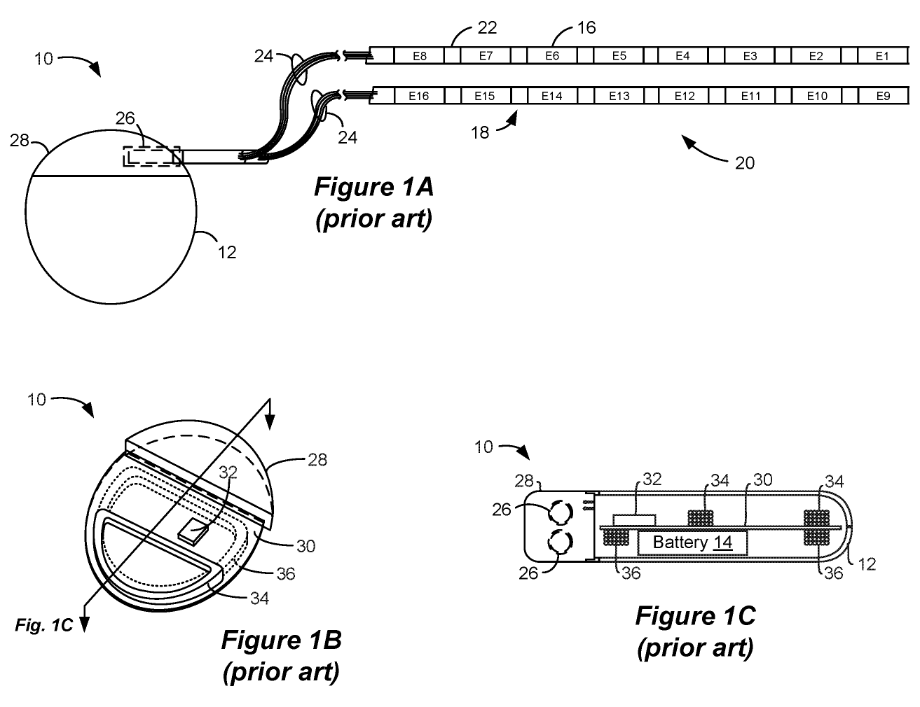

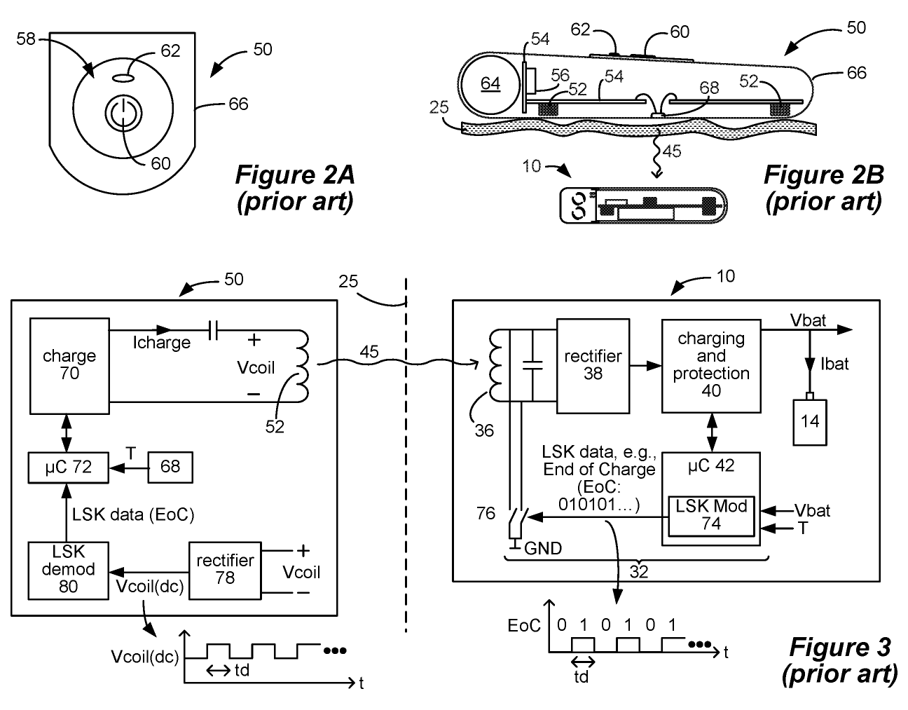

LED 132b may indicate the status of the battery 14 (FIG. 1C) in the IMD 10 being charged. In one example, implant battery LED 132b may not be controlled based on actual measurements of the capacity of the implant’s battery 14, but is instead controlled based on the charger 100’s operation. When the charger 100 is producing a magnetic field 145 and the charging coil assembly 102 is aligned with the IMD 10 being charged, the implant battery LED 132b can blink yellow to indicate that the implant battery 14 is being charged. Alignment between the charging coil assembly 102 and the IMD 10 is discussed further below. Once the implant’s battery 14 is full and the charger 100 receives telemetry from the IMD 10 of an end-of-charge (EoC) signal, this LED 132b can then be lit green. Alternatively, LED 132b may be controlled based on IMD battery 14 capacity measurements made in the IPG 10 (e.g., the voltage of battery 14). Such data can be telemetered from the IMD 10 to the charger 100.

One or more LEDs 134 may be controlled in unison to indicate the degree of coupling/alignment between the charging coil assembly 102 and the IMD 10 during a charging session, as explained further below. For example, LEDs 134 may be lit amber to indicate misalignment. Misalignment may also be indicated by not illuminating the LED of the button 130.

The charger 100 may also include a speaker 136 that emits tones or “beeps” in various circumstances. For example, the speaker 136 can emit beeps when the charging coil assembly 102 is misaligned with the IMD 10 being charged (in addition to also turning LEDs 134 amber). The speaker 136 may also issue a continuous double beep for a while (e.g., one minute) when the IMD’s battery 14 has been fully charged (once the charger receives the telemetered end-of-charge (EoC) signal). The speaker 136 may also beep a number of times when its battery 122 is critically low and thus the charger 100 is about to shut off (e.g., when LED 132a is red and blinking). The housing 120 may include openings comprising a speaker port 138 to facilitate sounds from the speaker 136 reaching the patient.

More complicated user interfaces, such as those incorporating a display, could also be provided with the electronics module 104, and as such, LEDs 132a, 132b, and 134 and button 130 could comprise indicators or touch-sensitive buttons on that display. User interface elements can be included on other faces of the electronic module’s housing 120, and may be placed such that they are easily viewed by the patient for the therapeutic application at hand (e.g., SCS, DBS).

Charging coil assembly 102 preferably contains only passive electronic components that are stimulated or read by the control circuitry 124 within the electronics module 104. Such components include the primary charging coil 150 already mentioned, which can comprise a winding of copper (e.g., Litz) wire that when energized by amplifier circuitry 200 (FIG. 7) in the electronics module 104 will create the magnetic field 145 that provides power to the IMD 10. As shown in FIG. 4B, the primary charging coil 150 is mounted to the top side of a circuit board 152 within a housing 154 of the charging coil assembly 102. Housing 154 is preferably formed of a plastic material (e.g., polycarbonate) and as shown preferably comprises a top housing portion 154a and a bottom housing portion 154b, which may be joined during manufacturing by screwing, snap fitting, ultrasonic welding, or solvent bonding. In one example, the charging coil assembly 102 contains no user interface elements, these instead being associated with the electronics module 104 as already described. However, the housing 154 of the charging coil assembly 102 may also have user interface elements (e.g., LEDs) to indicate statuses similar to those described with respect to LEDS 132a, 132b, and 134, or other indicators.

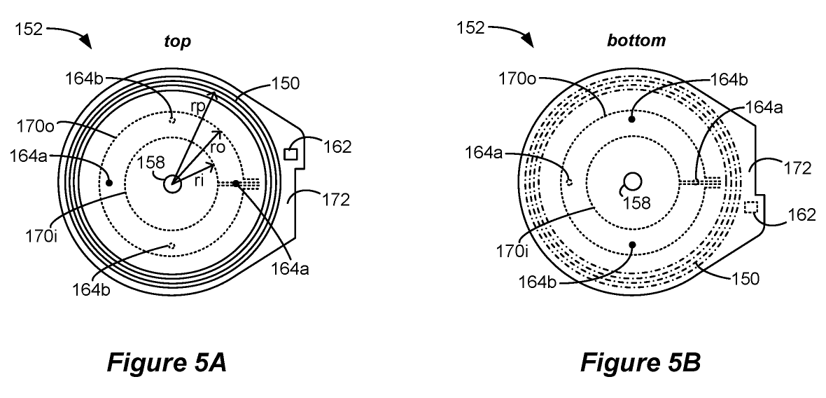

Various views of the circuit board 152 with housing portions 154a and 154b removed for easier viewing are shown in FIGS. 5A (top) and 5B (bottom). One or more bosses 156 (FIG. 4B) may be formed in the bottom housing portion 154b to fit within one or more holes 158 (FIGS. 5A and 5B) in the circuit board 152 to assist in affixing the circuit board in place within the housing 154. Top housing portion 154a may also have bosses or other stabilizing components, although this isn’t shown.

The circuit board 152 can be affixed in the housing 154 in other manners that are not shown, such as by snap fitting into clips formed into the bottom housing 154b. In another example, a thermally insulting material 160 (FIG. 4B) may be provided between the bottom of the circuit board 152 of the bottom housing 154b. This material 160 can comprise a foam tape having tacky surfaces on both of its sides to adhere to the bottom side of the circuit board 152 and to the inside surface of the bottom housing portion 154b. Preferably, this material 160 comprises a urethane foam, and more particularly a Poron™ urethane, manufactured by Rogers Corp. Use of a thermally insulating material 160 is preferred to prevent heat from the primary charging coil 150 from reaching the inside surface of the bottom housing portion 154b, which is most likely in contact with the patient during charging. Preferably, the thermal conductivity of thermally insulating material 160 is 0.2 W/m-K or less. In addition to providing thermal insultation, material 160 can help to dampen mechanical shock, thus preventing the charging coil assembly 102 from damage (e.g., if dropped). Material 160 is described in detail in U.S. Patent Application Publication 2018/0345025, which is incorporated herein by reference in its entirety.

Because the housing 154 of the charging coil assembly 102 is relatively thin—with a thickness x of 1.0 cm or less (FIG. 4B)—and because the primary charging coil 150 is generally located at the center of this thickness, the magnetic field 145 generated by the primary charging coil 150 during a charging session will generally be the same on both sides of the assembly. Therefore, and conveniently, either the top 154a or bottom 154b housing portion may face the patient (and the IMD 10) during a charging session.

Charging coil assembly 102 preferably includes at least one tuning capacitor 162 mounted to the circuit board 124. Capacitor 162 is coupled to the primary charging coil 150 (see FIG. 7) to tune the resonant frequency of this L-C circuit (e.g., to 80 kHz), and therefore to generally set the frequency of the AC magnetic field 145 (although this frequency is preferably adjustable, as explained further below). One skilled in the art will understand that the value of the capacitor 162 (C) connected to the primary charging coil 150 will be chosen depending on the inductance (L) of that coil and the desired frequency, in accordance with the equation f(res) = 1 / sqrt(2πLC). As one skilled in the art will appreciate, tuning capacitor 162 can be placed in series or in parallel with primary charging coil 126.

Also present in the charging coil assembly 102 are one or more temperature sensors 164, which are labeled 164a and 164b depending on whether such sensors are located on the top or bottom of the circuit board 152. As best shown in FIGS. 5A and 5B, two temperature sensors 136a are present on the top of circuit board 152, and two temperature sensors 136b are present on the bottom of circuit board, with each spaced 90-degrees within primary charging coil 150. In other examples, temperature sensors 164 may be present only on the top or only on the bottom of the circuit board 152. For example, only two temperature sensors 164a spaced at 180-degrees may be present on the top of the circuit board 152. The temperature sensors 164 may comprise thermistors, and in one example can comprise TMP112 High-Accuracy, Low-Power, Digital Temperature Sensors With SMBus and Two-Wire Serial Interface in SOT563, manufactured by Texas Instruments, Inc.

To assist with thermal management, and as explained in detail in the above-incorporated ‘025 Publication, the charging coil assembly 102 can further include a thermal diffuser 166 (FIG. 4B). In one example, the thermal diffuser 166 comprises a thermally conductive, soft plastic material, such as a non-silicone acrylic pad (e.g., Part No. 5590H, manufactured by 3M, Inc.), as discussed in the ‘025 Publication. Thermal diffuser 166 preferably has a tacky surface allowing it to be pressed onto and adhered to the top of the circuit board 152 and any components on this top surface, including temperatures sensors 164a. Thermal diffuser 166 preferably has a high thermal conductivity of greater than 1.0 W/m-K, and more preferably about 3.0 W/m-K. Thermal diffuser 166 also preferably has low electrical conductivity, and may have a dielectric constant of about 5-6 in one example. Thermal diffuser 166 preferably has a thickness greater than or equal to a thickness of the primary charging coil 150, which thickness may range from 0.5 to 4.0 mm for example. Because the thermal diffuser 166 is a soft material, it can be cut and shaped as necessary, as described in detail in the ‘025 Publication.

The thermal diffuser 166 is useful to dissipate heat away from the primary charging coil 150, which otherwise creates a hot ring-shaped area inside of the housing 154 when it is energized. Thermal diffuser 166 thus acts as a heat sink, and provides a heat transfer path away from the primary charging coil 150, thus distributing this heat over the diffuser’s larger circular area. Further, this distributed heat is directed to the temperature sensors 164a and/or 164b, particularly if in direct contact. As such, the temperature sensors are better able to accurately sense the temperature generated within the charging coil assembly 102.

To best distribute the primary charging coil 150’s heat, it is preferred that the thermal diffuser 166 be in contact with the primary charging coil, such as at an outer edge of the thermal diffuser, as shown in FIG. 4B. Although not shown, but as discussed in the ‘025 Publication, the thermal diffuser 166 may also overlap the primary charging coil 150. While it is beneficial to have the thermal diffuser 166 in contact with the primary charging coil 150, this is not strictly necessary. Heat may conduct from the primary charging coil 150 through the thermal diffuser 166 even if they are not in direct contact, as the heat may be transferred by intermediaries, such as the circuit board 152 or air within the charging coil assembly 102. In this regard, although not shown, the thermal diffuser 166 may be connected to one side of the circuit board 152 while the primary charging coil 150 is mounted to the other side.

As shown in FIGS. 5A and 5B, the charging coil assembly 102 further comprises inner and outer sense coils 170i and 170o. Generally speaking, these sense coils 170i and 170o are induced with a signal during generation of the magnetic field 145 from the primary charging coil 150. As discussed further below, the signals induced on the sense coils 170i and 170o are used for various purposes in the charger 100, such as to deduce alignment between the charging coil assembly 102 and the IMD 10, or to determine whether the power of the magnetic field 145 should be adjusted. The signal induced on the sense coils 170i and 170o can also be used to determine how to adjust the frequency of the magnetic field 145, and to receive back telemetry (e.g., LSK) from the IMD 10, as discussed further in the above-incorporated ‘350 Provisional Application. These induced signals are further affected by the underlying IMD 10 being charged, which is also inductively coupled to the primary charging coil 150.

Although the sense coils 170i and 170o could comprise wire-wound coils similar to the primary charging coil 150, in the disclosed example they are formed as traces in the circuit board 152. More specifically, these coils 170i and 170o preferably comprise a single trace turn, although they could be formed with multiple turns in the circuit board’s traces as well. The sense coils 170i and 170o are preferably concentric with the primary charging coil 150, although this isn’t strictly necessary. Furthermore, and like the primary charging coil 150, the sense coils 170i and 170o are preferably circular, although they could have different shapes as well (e.g., squares, etc.). As their names imply, the outer sense coil 170o has a radius ro larger than the radius ri of the inner sense coil 170i. Furthermore, both of these radii are preferably smaller than the radius rp of the primary charging coil 150 as shown. However, this isn’t strictly necessary. In other examples, the radius of the outer sense coil ro could be larger than the radius of the primary charging coil rp, with the radius of the inner sense coil ri being smaller than rp. Still further, the radii of both the outer and inner sense coils ro and ri could be larger than rp.

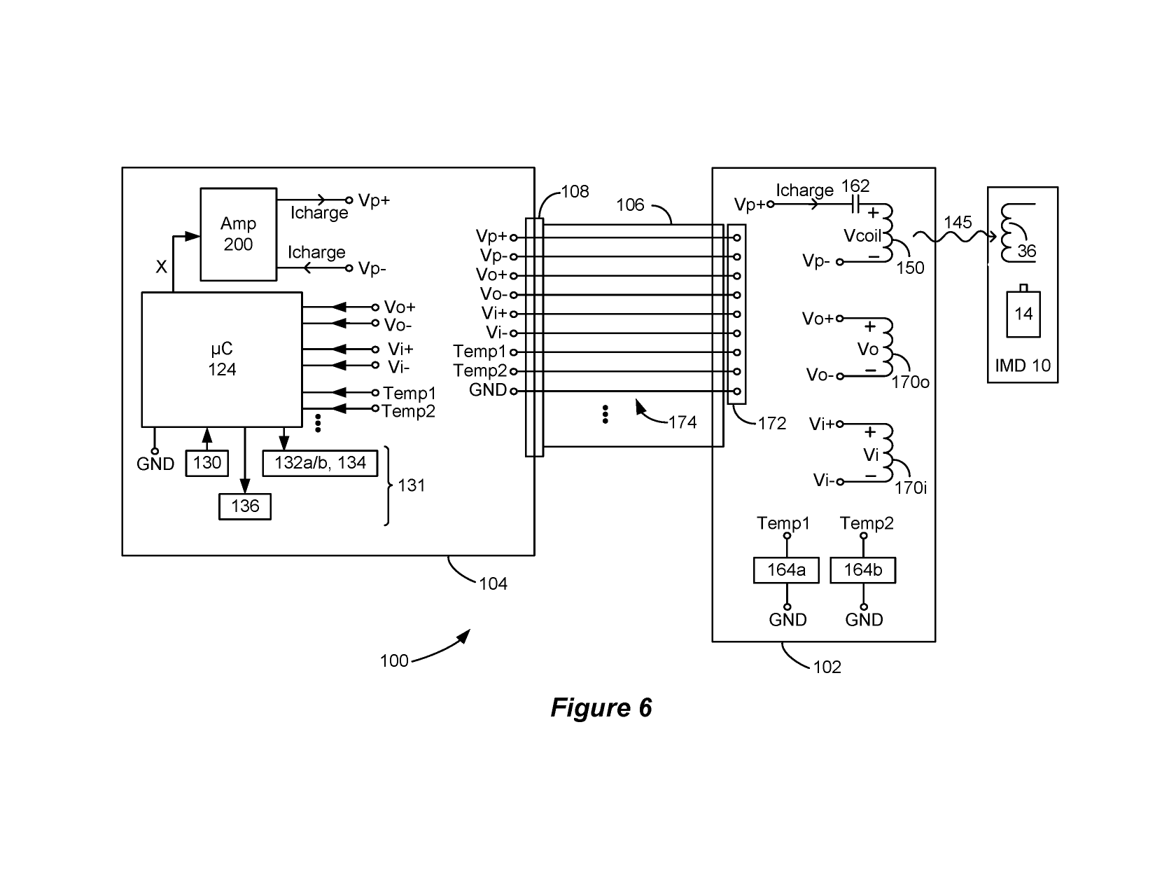

The circuit board 152 includes a contact portion 172 where wires 174 in cable 106 terminate and are connected to the components in the charging coil assembly 102, as best shown in FIGS. 4B and 6. One skilled in the art will understand that bond pads or similar contacts points would be present at the contact portion 172 to make these connections. In FIG. 6, connections are shown to both ends of the primary charging coil 150 (Vp+, Vp-); both ends of the outer sense coil 170o (Vo+, Vo-); both ends of the inner sense coil 170i (Vi+, Vi-); the temperature sensors 164 that are present (FIG. 6 assumes that two such sensors 164a and 164b are present, reporting two temperature Temp1 and Temp2); and a ground signal GND (as used e.g. by the temperature sensors 164). Although not shown, temperature data (Temp1 and Temp2) can be provided along a single I2C serial bus, and hence a single wire 174. Other wires 174 could also be included in the cable 106 as necessary to connect to other components in the charging coil assembly 102.

As shown in FIG. 6, signals from the sense coils 170i and 170o and the temperature sensors 164 are reported to the control circuitry 124 in the electronics module 104 and processed to useful ends, as discussed in more detail with respect to FIG. 7. As noted earlier, the charger 100 includes control circuitry 124 programmed via firmware with a number of functional modules whose functions are subsequently explained. These modules could also comprise discrete circuits, and thus need not necessarily comprise firmware programmed into control circuitry.

Temperatures reported by the sensors 164 (Temp1, Temp2) can be averaged by the control circuitry 124, and used to temporarily suspend the generation of the magnetic field 145 if the temperature gets too hot (above a maximum threshold). Once the temperature drops to a safe level (below a minimum threshold), the magnetic field 145 can again be started. As noted earlier, duty cycling the magnetic field 145 on and off this way based on temperature preferably occurs automatically and unbeknownst to the user.

As best shown in FIG. 7, the primary charging coil 150 is preferably driven using a class D amplifier 200, which is controlled by a digital drive signal X and its logical complement X*. This amplifier 200 has an H-bridge configuration comprising N-channel transistors 201a and 201b, and P-channel transistors 201c and 201d. When X is high (X* is low), transistors 201d and 201a are on and transistors 201b and 201c are off, thus causing current Icharge to flow through the primary charging coil 150 from constant power supply voltage Vcc to ground. When X is low (X* is high), transistors 201d and 201a are off and transistors 201b and 201c are on, thus causing current Icharge to flow through the primary charging coil 150 in the opposite direction. Therefore, Icharge is formed through the primary charging coil 150 as an AC current, which in turn produces magnetic field 145 as an AC field. For simplicity, drive signals X/X* are subsequently referred to singularly as drive signal X.

Drive signal X is output from pulse width modulator (PWM) 204, comprising firmware in the control circuitry 124. As shown, the drive signal X comprises a square wave with a particular on (a) / off (b) duty cycle. Programming on duration ‘a’ and off duration ‘b’ affects both the frequency f of the drive signal X (f = 1 / (a + b)) and the magnetic field 145. The frequency of the magnetic field 145 is nominally about 80 kHz, although this can vary in a range from 77 kHz to 87 kHz under control of a resonance module 216, as discussed further below.

Durations ‘a’ and ‘b’ also affect the duty cycle (DC = a / (a+b)) of the drive signal X, which adjusts the power of the magnetic field 145 under control of alignment and power module 210, as discussed further below. For example, a higher duty cycle (up to 50%) will create a larger AC current Icharge, thus creating a larger-magnitude AC magnetic field 145, and in turn allowing the battery 14 in the IMD 10 to be charged faster.

As noted above, the AC magnetic field 145 generated by the primary charging coil 150 will couple to and induce a signal in the outer and inner sense coils 170o and 170i, causing AC voltage Vo and Vi to build across each. Voltage Vo and Vi are also affected by coupling to the IMD 10. Voltages Vo and Vi are input to analog-to-digital (A/D) converters 203o and 203i to produce digital representations O and I of these waveforms. In the example shown, it is assumed that the control circuitry 124 includes (A/D) converters 203o and 203i at its inputs, but separate A/D circuitry could be provided as well if the control circuitry only includes digital inputs.

Signals O and I are continually determined when the charger 100 is producing the magnetic field 145. As explained further below, various other digital signals (Ao, Ai) dependent on O and I and produced from other modules in the control circuitry 124 are likewise also continually determined. This allows the charger 100 to make adjustments continually during an IMD charging session, as explained further below. One skilled will understand that continually in this context means determining O and I, and dependent signals, periodically at a suitably high frequency, such as every tenth of a second or so.

AC signals Vo and Vi (and hence digitized signals O and I) will vary in accordance with the coupling of the magnetic field 145 between the charger 100 and the IMD 10. More specifically, this coupling will vary in accordance with the coupling between the primary charging coil 150 in the charging coil assembly 102 and conductive structures in the IMD 10, including the secondary charging coil 36 and the IMD 10’s conductive case. This coupling is affected by the alignment (in x and y directions) between the primary charging coil 150 and the IMD 10 as well and the distance z between them.

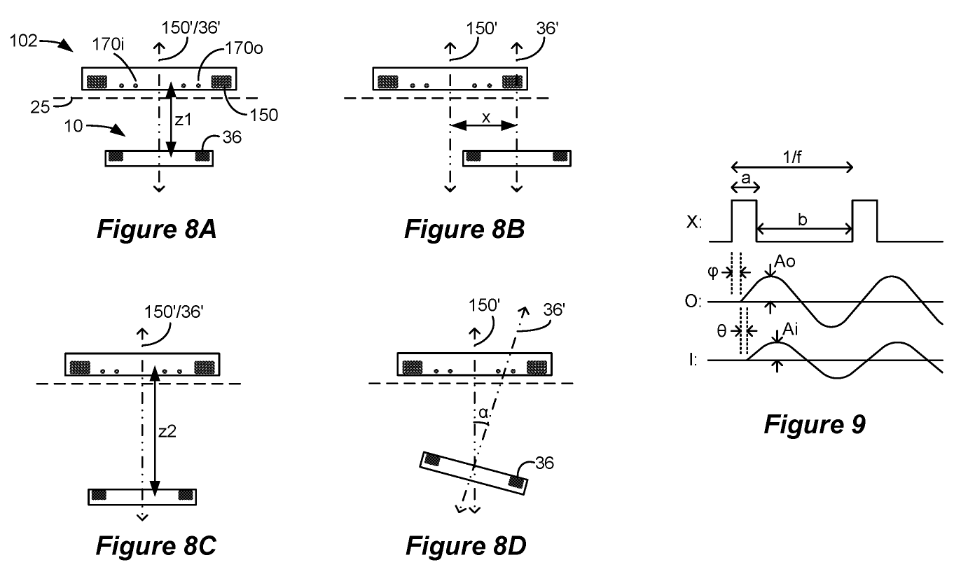

For example, the alignment is optimized between the primary charging coil 150 and the charging coil 36 in IMD 10 when the central axes of these coils 150’ and 36’ are collinear, meaning there is no x or y offset between the charging coils, as shown in FIG. 8A. By contrast, the alignment is poorer when these axes 150’ and 36’ are laterally shifted (in x and/or y), as shown in FIG. 8B. Poorer alignment implies poorer coupling, meaning that the magnetic field 145 isn’t as efficiently received by the IMD 14, lengthening the time it takes for the charger 100 to charge the battery 14. FIG. 8C shows the primary charging coil 150 and the IMD 10 to be well aligned, but the IMD 10 is implanted more deeply in the patient’s tissue 25. Compare z2 in FIG. 8C to z1 in FIG. 8A. Although aligned as best as possible, this also leads to poorer coupling. Coupling can also be negatively affected if the axes 150’ and 36’ are angled with respect to each other (α), which can occur for example if the IMD 10 is implanted at an angle in the patient (relative to the surface of the tissue 25), as shown in FIG. 8D. This is usually less significant however, as the IMD 10 as implanted and the charging coil assembly 102 are generally flat and relatively parallel.

In any event, the coupling between the primary charging coil 150 and the IMD 10 affects the amplitude of the AC signals induced on the sense coils 170o and 170i, as shown in FIG. 9. These induced signals as digitized (O, I) are shown in relation to the drive signal X. The signal O induced on the outer sense coil 170o has an amplitude Ao and a phase angle φ relative to the drive signal X. The signal I induced on the inner sense coil 170i has an amplitude Ai which is generally smaller than Ao because the inner sense coil 170i is farther from the primary charging coil 150 than is the outer sense coil 170o. Signal I, like O, has a phase angle relative to the drive signal X, and more specifically a phase angle θ relative to the outer induced signal O.

Amplitudes Ai and Ao are determined from digitized waveforms I and O at amplitude modules 212i and 212o (FIG. 7). Ai and Ao as determined by these modules preferably comprise maximum amplitudes for signals I and O as shown in FIG. 9, but could also comprise any DC voltage or parameter indicative of I’s magnitude. For example, amplitudes Ai and Ao can also be expressed as a (maximum) count (a number of steps) as output by the A/D circuitry (203o and 203i), where each count or step in the digital data is reflective of some voltage increment.

The charger 100 seeks to drive the frequency of the magnetic field 145 at the resonance frequency of the coupled charger/IMD system, and this occurs under control of the resonance module 216. Driving the primary charging coil 150 at resonance is preferred as this will improve the efficiency of receipt of the magnetic field 145 at the IMD 10. This results in less power loss, and faster charging of the IMD’s battery 14. The resonant frequency can be expected to vary over time during a charging session, for example, if the charging coil assembly 102 moves (e.g., x, y, z, or angle α) relative to the IMD 10 due to patient movement. As such, the resonance module 216 preferably continually varies frequency f of drive signal X during a charging session to compensate for this variance to maximize power transfer efficiency.

One manner in which the frequency can be adjusted in discussed in the above-incorporated ‘350 Provisional Application. This example involves monitoring the phase angle φ (FIG. 9) between the drive signal X and the outer sense coil 170o, whereby the frequency is adjusted until this phase angle is set to zero (or is otherwise minimized). However, this is merely one example in which the resonance module 216 can operate to set the frequency f. In another example, the primary coil 150 can be assessed directly, for example, by adjusting the frequency until the current through this coil is maximized.

The resonance module 216 adjusts the frequency f by periodically programming the PWM 204 with appropriate on and off times (a and b) for the drive signal X. In so doing, the resonance module 216 preferably does not adjust the power of the magnetic field 145 being produced (which is instead preferably and independently controlled by the alignment and power module 210 described below). As noted earlier, the power of the magnetic field 145 can be adjusted by adjusting the duty cycle of the drive signal X (a / (a+b)). To keep this duty cycle constant when varying the frequency f (1 / (a + b)), the resonance module 216 will adjust both a and b proportionally. For example, when the resonance module 216 decreases the frequency, both a and b are increased from their current values by the same percentage, which keeps the duty cycle (and hence the power) constant.

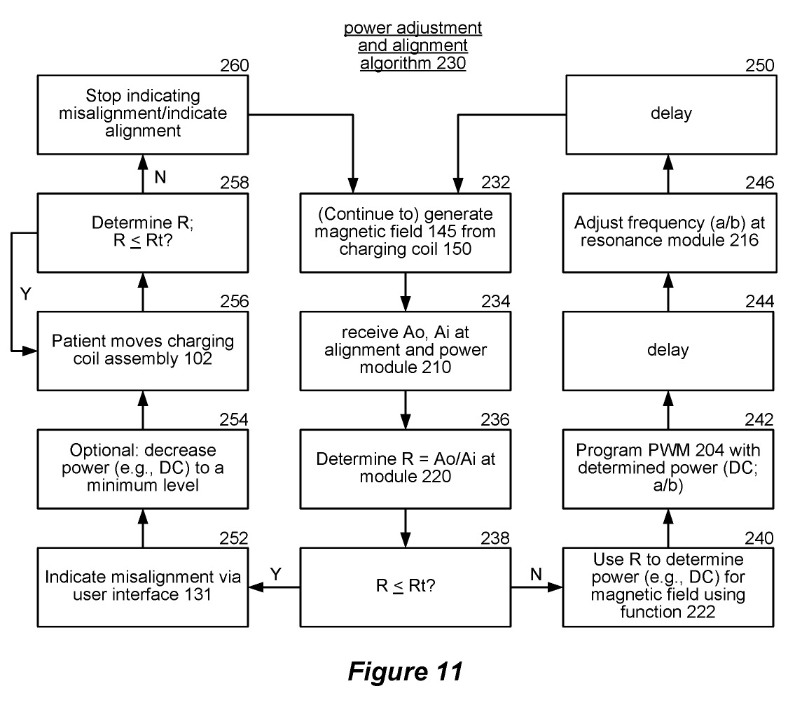

As shown in FIG. 7, the power of the magnetic field 145 can be adjusted using alignment and power module 210, which considers both the inner and outer amplitude Ai and Ao discussed earlier. In particular, module 210 includes a module 220 that determines a sense coil amplitude ratio R of Ao and Ai. As described, this ratio R equals Ao divided by Ai, but the reciprocal (R = Ai/Ao) could be determined and used considered as well. As described in detail below with reference to FIG. 11, a power adjustment and alignment algorithm 230 within module 210 uses this ratio R both to determine how to adjust the power of the magnetic field 145, and to determine whether the charging coil assembly 102 is aligned or misaligned with respect to the IMD 10. This algorithm 230 preferably queries a function 222 which relates different R values to different duty cycle (DC) values for the drive signal X, and hence to different powers for the magnetic field 145. Function 222 allows a duty cycle to be selected depending on the measured R value. Function 222 can comprise a mathematical function (e.g., DC = f(R)), or as shown can comprise a table that assigns a particular duty cycle to various R ranges.

Similarly to the resonance module 216, the alignment and power module 210 can adjust the power by periodically programming the PWM 204 with appropriate on and off times (a and b) for the drive signal X in accordance with a selected duty cycle (a / (a+b)). When adjusting the duty cycle (power), the module 210 preferably does not adjust the frequency of the magnetic field 145 being produced (which is instead preferably and independently controlled by the resonance module 216, as explained earlier). For example, if the module 210 needs to decrease the duty cycle to decrease the power of the magnetic field 145, it can decrease ‘a’ by an amount (a-Δ) but also increases ‘b’ by the same amount (b+Δ). Likewise, if the power module 216 needs to increase the duty cycle to increase the power, it can increase ‘a’ by an amount (a+Δ) but also decrease ‘b’ by the same amount (b-Δ). Adjusting a and b this way adjusts the duty cycle while preserving the (resonant) frequency established by the resonance module 216 (1 / (a + b)).

Note that both the resonance module 216 and the power module 214 can periodically adjust the on and off times (a and b) for the drive signal X during a charging session. To preserve the independence of these two modules, and to prevent conflicts, it is preferred that the resonance module 216 and the power module 214 not adjust these parameters at the same time. Instead, it is preferred to interleave the adjustments made by these modules 216 and 214 in time. This is explained further below with reference to FIG. 11.

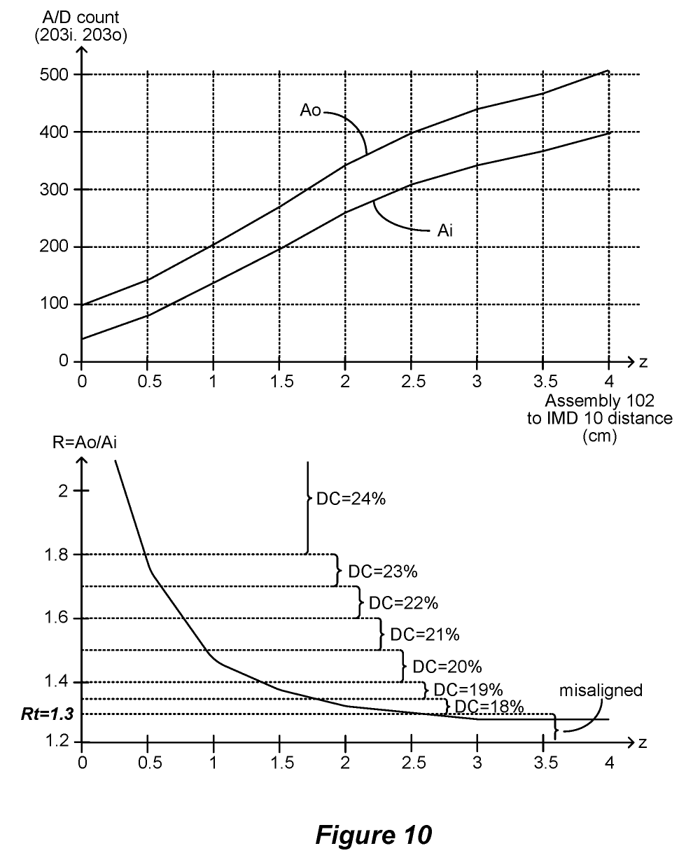

FIG. 10 shows experimental data explaining the relevance of the sense coil amplitude ratio R to adjusting power of the magnetic field 145, and to determining alignment. The top graph shows raw values for the amplitudes Ao and Ai induced on the outer and inner sense coils 170o and 170i. In the depicted example, these amplitudes are expressed as a number of counts or steps of the A/D circuitry (203o and 203i), but could also comprise voltage values as well. These amplitudes Ao and Ai were determined by placing the IMD 10 at different distances (depths z; see, e.g., FIGS. 8A and 8C) with respect to the charging coil assembly 102, but with that assembly in good alignment with the IMD (i.e., no x or y offset; compare FIG. 8B). A constant power for the magnetic field was used (a duty cycle of 20%), and the frequency of that field was adjusted to resonance (e.g., using resonance module 216) at each tested distance.

As expected, the outer amplitude Ao is higher than the inner amplitude Ai owing to the outer sense coil 170o being more proximate (and hence better coupled) to the primary charging coil 150. Furthermore, as the distance (z) increases, these amplitudes Ao and Ai also increase. This is due to back EMF formed by the IMD 10 (i.e., an opposing magnetic field that is generated in the IMD 10 in response to the magnetic field 145). This back EMF is stronger if the IMD 10 is closer (and hence better coupled) to the charger 100, and reduces the amplitudes of the induced voltages on the sense coils. By contrast, this back EMF is weaker if the IMD 10 is farther away, and thus the voltages on the sense coils will increase because coupling with the primary sense coil 150 is not as strongly counteracted by the back EMF.

The ratio R between amplitude Ao and Ai is shown in the bottom graph in FIG. 10, which illustrates that the relative difference between these amplitudes is more pronounced at shorter distances (z), and less pronounced at longer distances. This is due to the inner sense coil 170i being more sensitive to back EMF from the IMD 10 compared to the outer sense coil 170o. Because the inner sense coil 170i is farther from, and therefore less affected by, the primary charging coil 150, it is better able to “pick up” the back EMF from the IMD 10. As already noted, this back EMF is more prominent at shorter distances (z), and thus a greater relative difference between Ao and Ai (i.e., a larger R) results at these shorter distances. By contrast, at longer distances, this back EMF becomes less prominent, and thus the ratio R asymptotically decreases towards a more constant value (e.g., R ~ 1.28) reflective solely of the coupling of each sense coil to the primary charging coil 150.

As already noted, ratio R can be used to adjust the power of the magnetic field 145, and further to determine whether the charging coil assembly 102 and IMD 10 are aligned or misaligned. As concerns power adjustment, power data (duty cycles) appropriate for each R value (or ranges of R values) are shown in the bottom graph of FIG. 10B, as taken from function 222. This power data is preferably designed to ensure that the IMD 10 will receive a suitable (ideally, constant) amount of power from the magnetic field 145, thus ensuring that the IMD 10 will be adequately charged regardless of its distance (and coupling) to the charging coil assembly 102.

As shown, higher values for R—which indicate a smaller distance between the charging coil assembly 102 and the IMD 10—warrant the use of higher duty cycles: for example, if R is between 1.7 and 1.8 (because the IMD is relatively close), a duty cycle of 23% is indicated, whereas if R is between 1.35 and 1.4 (because the IMD is relatively close), a duty cycle of 19% is indicated. As such, function 222 proportionately relates ratios to duty cycles (although not necessarily linearly), with higher ratios associated with higher duty cycles and lower ratios associated with lower duty cycles. This seems counterintuitive: if the IMD 10 is closer to and generally better coupled to the charging coil assembly 102, it will more efficiently receive power from the magnetic field 145, which might suggest that the power (the duty cycle) could be lowered. However, the back EMF from the IMD 10 is also more prominent at shorter distances, which also affects the primary charging coil 150, increasing the reflected impedance experienced by that coil. As a result, it is preferable to drive the primary charging coil 150 harder (with a higher duty cycle) to compensate and to provide more power when R is low. By contrast, if the IMD 10 is relatively deep, this will decrease the reflected impedance experienced by the primary charging coil 150, which need not be driven as hard to deliver suitable power to the IMD 10. As such, when R is lower, the duty cycle can be decreased.

Alignment can also be determined using ratio R. Although the data shown in FIG. 10 assumes that the charging coil assembly 102 and IMD 10 are perfectly aligned, this data more generally illustrates the effect of coupling between charging coil assembly 102 and IMD 10. In this regard, a deeply implanted but aligned IMD 10 (e.g., FIG. 8C) and a shallowly implanted IMD 10 that are misaligned (e.g., FIG. 8B) are generally both poorly coupled to the charging coil assembly 102. This poor coupling (regardless of the reason) is reflected by lower values for R. As such, a threshold value for R (Rt) can be set in the alignment and power module 210, with values of R lower than this threshold indicating misalignment, and higher values indicating reasonable alignment. As illustrated, this alignment threshold Rt is set to an R value of 1.3.

Notice from FIG. 10 that this value for the alignment threshold Rt=1.3 generally corresponds to an aligned implant depth of about 2.7 centimeters. This threshold value would thus not be suitable for determining alignment when the IMD 10 is implanted more deeply than a distance of 2.7 cm from the charging coil assembly 102, because R would always be less than this value, even when the alignment is ideal. Nevertheless, most IMDs 10 are not implanted so deeply, and instead can be expected to have a distance of no more than 2 centimeters from the charging coil assembly 102. As such, this alignment threshold Rt is generally suitable to determine alignment from misalignment for the described charger 100/IMD 10 system. Of course, the particular value chosen for the alignment threshold Rt can be varied in different implementations.

While values for ratio R are noticed to be heavily dependent on positing of the charging coil assembly 102 relative to the IMD 10 (coupling) as just discussed, empirical data also suggests that R is relatively independent of the duty cycle and frequency used to produce the magnetic field 145, at least given normal ranges in which these parameters would normally be adjusted. This is beneficial, as this allows the duty cycle and frequency to be changed during a charging session in manners that would not affect R. Such duty cycle and frequency adjustments can therefore be made in the power adjustment and alignment algorithm 230, discussed next, without significantly affecting the integrity of R as a feedback variable.

FIG. 11 shows an example of the power adjustment and alignment algorithm 230, which uses the sense coil amplitude ratio R to both adjust power and to determine misalignment. A magnetic field 145 is generated at the charger 100 at step 232, which may first occur when the user presses button 130 of the user interface 131 (FIG. 4B). The generation of magnetic field 145 induces signals on the sense coils 170o and 170i with amplitudes Ao and Ai as discussed earlier. These amplitudes are received at the module 210 at step 234, and their ratio R is computed at module 220 at step 237.

At step 238, the determined ratio value R is compared to the alignment threshold Rt discussed earlier. If R is less than or equal to Rt, the algorithm 230 determines that the charging coil assembly 102 is misaligned with the IMD 10. Thus, at step 252, misalignment can be indicated at the charger 100’s user interface 131. As discussed earlier, indicating misalignment can be accomplished for example by illuminating LEDs 134 (e.g., amber), by not illuminating the LED of the button 130, by issuing beeps from the speaker 136, or by taking all of these actions.

As an optional step at 256, the algorithm 230 can decrease the power (e.g., the duty cycle) of the magnetic field 145 when the charging coil assembly 102 is misaligned for safety reasons (e.g., to prevent overheating). Preferably, decreasing the power can comprise decreasing the power to a minimum level. In one example, this minimum power can comprise a duty cycle of 15%. The minimum power level is preferably one at which the algorithm 230 still generates a magnetic field 145 and can still operate to reliably determine ratio R based on induced signals Ao and Ai, and hence determine that the charging coil assembly 102 is eventually brought back into alignment. But again, step 254 is optional, and instead the algorithm 230 can continue using the power level as previously determined by the algorithm 230 (as described further below).

Regardless whether the power has been decreased at this point, once misalignment has been indicated to the user (252), the user can at step 256 attempt to move the charging coil assembly 102 into better alignment with the IMD 10. As this occurs, the algorithm 230 continues at step 258 to monitor R (by receiving updated Ao and Ai values). If at this step 258, R continues to be less than or equal to Rt, the algorithm 230 continues to monitor R as the user continues to move the charging coil assembly 102 (256). Eventually when the charging coil assembly 102 is moved into proper alignment, R will be greater than Rt at step 258. Because there is no longer misalignment, any misalignment indications issued at the user interface 131 earlier (252) can cease at step 260 (e.g., LEDs 134 can be unlit, and speaker 136 will be silenced), and/or alignment can be indicated (e.g., by light button 130’s LED green).

The algorithm 230 can then continue generating the magnetic field 145 at step 232, determine a new value for R at step 236. Assuming at step 238 that R is still greater than Rt and therefore that charging coil assembly 102 and IMD 10 are in reasonable alignment, the algorithm 230 can proceed to adjust the power of the magnetic field 145 at step 240. As discussed earlier, this occurs using function 222, which determines a power (more specifically a duty cycle DC) appropriate for the currently-reported R value. This determined duty cycle is then programmed at the PWM 204 at step 242, which as noted earlier can involve adjustment of the ‘a’ (on) and ‘b’ durations of the drive signal X.

After a delay at step 244, the frequency can be adjusted as necessary by the resonance module 216 at step 246, which can also involve adjustment of the ‘a’ (on) and ‘b’ durations of the drive signal X at the PWM 204. As discussed earlier, this is desirable to bring the frequency of the drive signal X and the magnetic field 145 to resonance. While it is desirable to adjust the frequency to resonance, this is also not strictly necessary, and thus step 246 can be considered optional. Instead, the frequency can be adjusted in other ways not involving the use of the power adjust and alignment algorithm 230, or the frequency could simply be held constant.

After another delay at step 250, the algorithm 230 can return to step 232, where the magnetic field 145 continues to be generated (232) and R assessed (steps 234-236), to determine later during the charging session if there is misalignment (238), and if not whether the power should be adjusted further (240), etc.

Delays 244 and 250 are preferred to ensure that the algorithm 230 doesn’t make adjustments unnecessarily frequently. These delays 244 and 250 can be of the same or different durations, and may be on the order of tenths of a second. Delays 244 and 250 are also useful to separate the power (240, 242) and frequency (246) adjustments, and to interleave them in time. This is beneficial to reduce conflicts between these two steps, both of which can involve changing the ‘a’ (on) and ‘b’ durations of the drive signal X at the PWM 204. Further, and as discussed earlier, these a/b adjustments are preferably intelligently made in a manner where the duty cycle/power can be changed without affecting the frequency, and vice versa.

Algorithm 230 is described as preferably being able to adjust power and determine misalignment using sense coil amplitude ratio R. However, this is not strictly necessary, and instead algorithm 230 could be modified to perform the functions of power adjustment and misalignment determination individually. For example, algorithm 230 could modified to only provide for power adjustment. In this example, steps 238 (comparison of R to an alignment threshold Rt) and steps 252-260 could be omitted; if necessary, misalignment could be determined in the charger 100 using different means not involving the use of ratio R. Algorithm 230 could also be modified to only determine misalignment. In this example, steps 240-250 relevant to power adjustment could be omitted; again, if necessary, power adjustment could be performed in the charger 100 using different means not involving the use of ratio R.

As disclosed and described, computation of a ratio R (e.g., Ao/Ai) is used in the alignment and power module 210 and in the power adjustment and alignment algorithm 230 to determine alignment and/or to adjust the power. However, it is not strictly required that a ratio be taken of variables Ao and Ai. Instead, a different parameter (P) can be determined using Ao and Ai and used in module 210 and algorithm 230 in lieu of R. This different parameter P can comprise a function of Ao and Ai, and possibly of other variables (e.g., φ or θ) or constants (e.g., k) as well (e.g., P = Ao²/Ai; P = Ao / (Ai+k); P = cos(φ) * (Ao/Ai); etc.).

Referring again to FIG. 7, telemetry from the IMD 10 can be processed in the control circuity 124 by use of an LSK demodulator 202. This LSK demodulator 202 can assess the voltage on the primary charging coil 150 similar to what was described earlier (see FIG. 3). The LSK demodulator 202 can alternatively assess various signals associated with the sense coils 170o and 170i, as described in further detail in the above-incorporated ‘350 Provisional Application. Regardless of the implementation, the LSK demodulator 202 can receive LSK data from the IMD 10, such as an End-of-Charge signal, or other LSK data that might be useful to the charger 100 when controlling charging. Such additional LSK data can include for example, the IMD’s charging current Ibat, the voltage of the IMD’s battery 14 Vbat, or a temperature in the IMD.

In place of, or in addition to, use of LSK telemetry, the charger 100 can include short-range RF telemetry means for communicating with the IMD 10. Thus, the charger 100 and IMD 10 could both include short-range RF antennas and associated modulation/demodulation chip sets (not shown) to allow communications using far-field electromagnetic waves. Such communications could occur using the well-known Bluetooth standard for example. As such, the IMD 10 can provide telemetry (the EoC signal, or other charging parameters or control signals) to the charger 100 using such means.

Although charger 100 is disclosed as being useful to wirelessly charger an IMD 10, one skilled will understand that charger 100 could be used to wirelessly charge any device, including those that are not implantable or that are not medical devices.

While the charger 100 is described above as having two sense coils—an outer and an inner sense coil 170o and 170i—this is not strictly necessary. Instead, one, or three or more, sense coils could be used. If three or more sense coils are used in the charger 100, preferred and most sensitive of the sense coils could be used in the charger to determine various conditions. For example, a first sense coil amplitude ratio between a first and second sense coil could be used for power adjustment, while a second sense coil amplitude ratio between a first and third sense coil could be used to determine alignment.

Although examples of the charger 100 are described as being used in implantable stimulation devices system such as a spinal cord stimulation system, the antennas, circuitries, and algorithms described herein may be used in other implantable medical device systems to charge other types of implantable medical devices. For example, in some implementations, the implantable medical device may include an electric pump that is configured to move fluid within the implantable medical device to inflate or deflate an inflatable member. For example, in some implementations, the implantable medical device may be an inflatable penile implant or an inflatable artificial sphincter (such as an artificial urinary sphincter). In some cases, inflatable penile implants are used to help address erectile disfunction issues. In some cases, an inflatable artificial sphincters are used to help address continence issues.

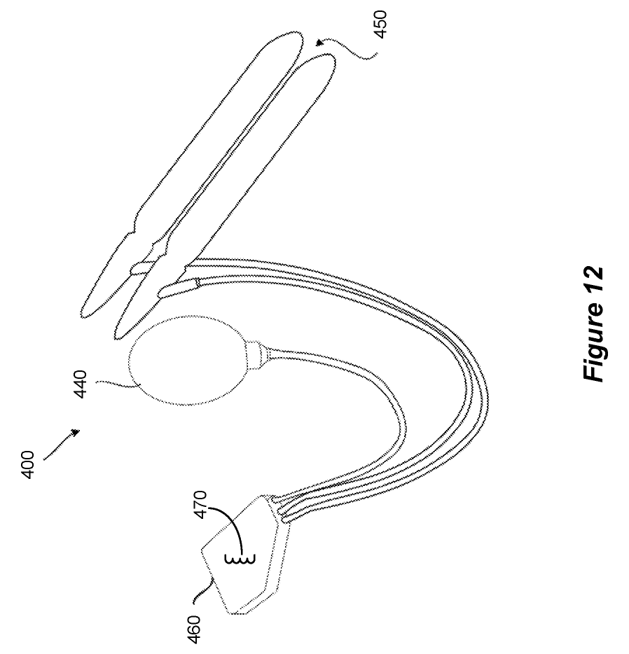

As illustrated in FIG. 12, an inflatable penile implant 400 (a type of implantable medical device, IMD) includes a fluid reservoir 440 configured to be placed within a pelvic region of a patient and one or more inflatable members 450 configured to be placed within a penis of the patient. The inflatable penile implant 400 also includes a housing 460 (including a case and a header) that is operatively coupled to the fluid reservoir 440 and to the inflatable member(s) 450. The housing 460 may house a power source (such as a rechargeable battery) and a pump or pumps (such as a piezo-electric pump or pumps) configured to move fluid to and from the inflatable member(s) 450 to place them in an inflated configuration or a deflated configuration. The housing 460 may also house an antenna or coil 470 analogous to secondary charging coil 36 (FIG. 3) to allow the inflatable penile implant 400 to receive the magnetic field 145 from the charger 100, thus providing power for the inflatable penile implant 400 and allowing its battery to be charged.

As discussed earlier, the various algorithms and modules described herein can be implemented as firmware or software, and such algorithms, firmware, or software may be embodied in a non-transitory computer readable media, such as a solid-state memory (e.g., control circuitry 124 in the charger 100), optical or magnetic disks, and the like. These media may be present outside of the charger 100, and stored in manners downloadable to the charger 100, such as on various Internet servers, portable or stationary disks, manufacturing computer systems, and the like.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An external charger for wirelessly providing energy to an implantable medical device (IMD), comprising:
a charging coil configured when energized to produce a magnetic field to wirelessly provide energy to the IMD during a charging session;
a first sense coil, wherein the first sense coil is configured to be induced by the magnetic field with a first induced signal;
a second sense coil, wherein the second sense coil is configured to be induced by the magnetic field with a second induced signal; and
control circuitry configured to
determine a first amplitude of the first induced signal and a second amplitude of the second induced signal,
divide the first amplitude by the second amplitude to determine a ratio, and
use the ratio to adjust a power of the magnetic field.

2. The external charger of claim 1, wherein the control circuitry is further configured to determine an alignment between the charging coil and the IMD using the ratio.

3. The external charger of claim 2, wherein the control circuitry is configured to determine the alignment by determining if the charging coil and the IMD are aligned or misaligned by comparing the ratio to a threshold.

4. The external charger of claim 3, wherein the control circuitry is configured to determine that the charging coil and the IMD are misaligned when the ratio is less than the threshold.

5. The external charger of claim 3, further comprising a user interface comprising one or more user interface elements, wherein if the control circuitry determines that the charging coil and the IMD are misaligned, the control circuitry is further configured to enable one or more of the user interface elements.

6. The external charger of claim 1, further comprising a circuit board comprising the charging coil, the first sense coil, and the second sense coil.

7. The external charger of claim 6, wherein the first and second sense coils comprise one or more traces in the circuit board.

8. The external charger of claim 6, wherein the charging coil, the first sense coil, and the second sense coil are concentric.

9. The external charger of claim 8, wherein the first sense coil has a radius larger than a radius of the second sense coil.

10. The external charger of claim 9, wherein the charging coil has a radius larger than the radii of the first and second sense coils.

11. The external charger of claim 1, wherein the control circuitry is configured to determine the ratio periodically during the charging session, and thus periodically use the ratio to adjust the power of the magnetic field.

12. The external charger of claim 1, wherein the external charger comprises a first housing comprising the charging coil and the first and second sense coils, a second housing comprising the control circuitry, and a cable for passing signals between the first and second housings.

13. The external charger of claim 1, further comprising an amplifier, wherein the amplifier is configured to energize the charging coil to produce the magnetic field in accordance with a drive signal.

14. The external charger of claim 13, wherein the drive signal comprises a duty cycle, and wherein the control circuitry is configured to use the ratio to adjust the power of the magnetic field by using the ratio to adjust the duty cycle of the drive signal.

15. The external charger of claim 14, wherein the control circuitry comprises a pulse width modulator to adjust the duty cycle of the drive signal.

16. The external charger of claim 14, wherein the control circuitry further comprises a function relating ratios to duty cycles, and wherein the control circuitry is configured to adjust the duty cycle using the ratio and the function.

17. The external charger of claim 16, wherein the first sense coil is larger than the second sense coil, and wherein the function proportionately relates the ratios to the duty cycles.

18. The external charger of claim 1, wherein the first and second induced signals are AC signals, and wherein the first and second amplitudes are respectively indicative of maximum amplitudes of the first and second induced signals.

19. A method for wirelessly providing energy to an implantable medical device (IMD), the method comprising:
energizing a charging coil in an external charger to produce a magnetic field to wirelessly provide energy to the IMD during a charging session;
inducing by the magnetic field a first induced signal on a first sense coil in the external charger;
inducing by the magnetic field a second induced signal on a second sense coil in the external charger;
determining a first amplitude of the first induced signal and a second amplitude of the second induced signal;
dividing the first amplitude by the second amplitude to determine a ratio; and
using the ratio to adjust a power of the magnetic field.

20. A computer readable medium comprising instructions executable in an external charger for wirelessly providing energy to an implantable medical device (IMD), wherein the instructions when executed are configured to:
energize a charging coil in the external charger to produce a magnetic field to wirelessly provide energy to the IMD during a charging session;
determine a first amplitude of a first induced signal induced on a first sense coil in the external charger and a second amplitude of a second induced signal induced on a second sense coil in the external charger;
divide the first amplitude by the second amplitude to determine a ratio; and
use the ratio to adjust a power of the magnetic field.
